(19) United States
(12) Patent Application Publication
(10) Pub. No.: US 2026/0215976 A1
(43) Pub. Date: Jul. 30, 2026

(54) ABSORBENT ARTICLE WITH CELLULOSIC STRUCTURES

(71) Applicant: Nicole Richards, Madison, NC (US)

(72) Inventor: Nicole Richards, Madison, NC (US)

(21) Appl. No.: 19/037,548

(22) Filed: Jan. 27, 2025

(51) Int. Cl. A61F 13/514; A61F 13/534

(52) U.S. Cl. CPC A61F 13/51474; A61F 13/51405; A61F 13/534

(57) ABSTRACT

Absorbent articles, such as diapers, sanitary pads, incontinence products, and similar articles can utilize cellulosic structures as a sustainable replacement for conventional polymers, delivering performance comparable to plastic-based materials. These articles can incorporate a cellulosic top sheet and/or back sheet with a hydrophobic treatment. They may also incorporate cellulosic materials for acquisition and distribution layer, core wrap, and/or core.

### COPYRIGHT AND TRADEMARK STATEMENT

A portion of the disclosure of this patent document may contain material that is subject to copyright protection. The copyright owner(s) has no objection to the facsimile reproduction by anyone of the patent document or the patent disclosure as it appears in the Patent and Trademark Office patent file or records, but otherwise reserves all copyright rights whatsoever. Trademarks may be used in the present disclosure, and the applicant(s) make no claim to any trademarks referenced.

### FIELD OF THE INVENTION

The present disclosure pertains to absorbent articles, such as diapers, sanitary pads, or incontinence products, and more particularly to an absorbent material for use in such article.

### BACKGROUND OF THE INVENTION

Absorbent articles designed to manage waste body fluids such as urine, blood, and menstrual discharge are widely used across the globe. Commonly referred to as “absorbent articles,” these products typically include items like diapers, feminine hygiene products, and incontinence products. Over time, these articles have become essential for modern convenience and hygiene.

Diapers, a widely used absorbent article, are classified as reusable cloth or disposable types. Modern cloth diapers often include liners, such as flushable ones, to retain waste for easy disposal. Disposable diapers became dominant after the mid-1980s introduction of superabsorbent polymers (SAP) like sodium polyacrylate and polyacrylamide copolymers. Currently, about 90% of U.S. parents use disposable diapers for their children.

A standard disposable diaper consists of several key components:
(a) Inner Layer (Top Sheet): This layer rests directly against the baby's skin and serves as the initial contact point for waste fluids and solids. Traditionally, this layer is made from petroleum-based plastic or plastic-treated materials. However, some eco-friendly diaper manufacturers are now using plant-based plastics (bioplastics) or hydrophilic cotton blends for this purpose.
(b) The Absorption Distribution Layer (ADL), beneath the top sheet, channels and evenly distributes fluids into the absorbent core. Composed of hydrophilic polypropylene with lightweight fiber layers and air pockets, it enhances fluid movement, prevents pooling, and ensures rapid, uniform absorption.
(c) The absorbent core, the innermost and thickest layer, is enclosed in a core wrap and made of cellulose fluff pulp. It absorbs and transfers fluid to superabsorbent polymer (SAP), with the SAP granules forming a gel upon absorbing liquid, effectively locking fluids away from the baby's skin.
(d) Waterproof Outer Shell: This outer layer (Back sheet) commonly made from petroleum-based plastic or plastic-treated materials, prevents leaks and provides a softer, more comfortable external surface. Some sustainable brands use plant-based plastics (bioplastics) or nonwoven fabrics as eco-friendly alternatives. In certain designs, an additional semi-outer film layer may enhance waterproofing and durability.

Renewable materials, such as cotton, bamboo (viscose), hemp, wood pulp cellulose, and plant-derived plastics like “green” polyethylene, polypropylene, and PLA, are increasingly used in absorbent article components, particularly in top sheets, liners, and back sheets. These natural fibers, valued for their high cellulose content and absorbent properties, are driven by consumer demand. However, they can retain moisture, potentially causing skin irritation if such moisture is not efficiently transferred away from the skin.

### SUMMARY OF THE INVENTION

Bearing in mind the problems and deficiencies of the prior art, it is therefore an object of the present invention to provide absorbent articles which incorporate renewable cellulosic materials while providing performance comparable or better than articles fabricated from non-sustainable materials.

An absorbent article suitable for use as diapers, nappies, absorbent underpants, training pants, adult incontinence products, pet incontinence products, feminine hygiene products, wound dressings, breast pads, and any other similar products may include at least one layer that comprises a fabric of cellulosic fibers treated with one or more hydrophobic treatments, such treatment(s) being selected from the group consisting of:
blending the cellulosic fibers with synthetic fibers,
leaving at least a portion of the cellulosic fibers unbleached so as to leave inherent lignin present, and
applying a hydrophobic agent to at least one surface of the cellulosic fibers (such agent being applied to at least one of a surface of the cellulosic fibers and a surface of the fabric formed therefrom).

Where the hydrophobic treatment comprises applying a hydrophobic agent, such agent may be applied to the fibers and/or to the fabric formed therefrom, and may comprise at least one compound selected from the group consisting of: silica-based nanoparticles; graphene-based coatings; TiO₂ (titanium dioxide) coatings; Zinc oxide and zinc pyrithione coatings; copper oxide, copper gluconate, and copper sulfate coatings; epoxies; nanomaterial-based resins, polymers, and surfactants; Lotus effect-inspired coatings; Alkyl-based coatings; polymeric films from 1-120 μm; Self-healing hydrophobic coatings; functionalized polymers with hydrophobic side chains, such as alkyl groups; hydrophobic highly branched dendrimers; paraffins that are liquid at room temperature in water dispersible emulsions; and fatty acids.

The layer may form the inner layer of the article, that is, the layer that rests against the skin of the user when the article is in use (“top sheet”). The layer may form the outer layer of the article, that is, the layer that faces outwards from the skin of the user when the article is in use (‘back sheet”). Both the inner layer and the outer layer could each comprise a fabric of cellulosic fibers treated with one or more hydrophobic treatments as described.

In some embodiments, the hydrophobic treatment comprises applying one or more of applying a fatty acid and/or a fatty acid emulsion, and/or a paraffin wax and/or a paraffin wax emulsion to at least one surface of the cellulosic fibers, and/or applying a hydrophobic agent to staple cellulosic fibers used to form the fabric prior to fabric formation (such application being at less than 5% by weight add on).

The absorbent article may further comprise a second layer and a core interposed between the layers, and wherein the layers and the core are free from oil based microplastic-emitting materials and petrochemical derivatives.

The absorbent article may have a total carbon content greater than 50, a molecular number less than 320 and molecular weight less than 900.

The absorbent article may have a semi-outer layer wherein the semi-outer layer has an MVTR rating of at least 600 g/m²/day.

When the layer of cellulosic fabric is an inner layer that rests against the skin of the user when the article is in use, the hydrophobic treatment may comprise applying a hydrophobic agent to only a top surface of the inner layer (that is, the surface facing towards the skin of the wearer when the article is in use). The hydrophobic treatment may comprise applying a hydrophobic agent to the inner layer at 0.009% to 1.75% by weight add on. The inner layer may be configured to achieve a strike-through rate of less than 8 seconds and a rewet value of less than 40 grams as per INDA standardized testing, with sterile solution of sodium chloride (salt) in water at a concentration of 0.9% NaCl (standardized saline concentration). The absorbent article may further comprise a hydrophobic nonwoven outer layer comprising cellulosic nonwoven fibers and a semi-outer film layer, where the outer layer and the film layer, in combination with the inner layer, provide the article with a strike-through rate of less than 8 seconds and a rewet value of less than 40 grams as per INDA standardized testing, with sterile solution of sodium chloride (salt) in water at a concentration of 0.9% NaCl (standardized saline concentration. At least one surface of the inner layer may comprise a plurality of pores. The absorbent article may further comprise an ADL layer that comprises cellulosic fibers, and/or an absorbent core layer that is encapsulated in cellulosic fibers. The absorbent article may further comprise a semi-outer film layer that has a MVTR breathability rating of 600 g/m²/day or greater and/or an outer layer that comprises cellulosic fibers.

When the layer of cellulosic fabric is an outer layer that faces outwards from the skin of the user when the article is in use, the hydrophobic treatment may comprise applying a hydrophobic agent to the outer layer at 0.05% to 6.25% by weight add on. The outer layer may have an MVTR rating of at least 600 g/m²/day. The outer layer may be configured to achieve a vertical wicking capability of less than 70 mm using AATCC TM197-2022 testing and a water repellency rating above 75 using AATCC TM22 testing.

An absorbent article may comprise an inner layer that rests against the skin of the user when the article is in use, said inner layer comprising a fabric of cellulosic fibers treated with one or more hydrophobic treatments selected from the group of:
blending the cellulosic fibers with synthetic fibers,
leaving at least a portion of the cellulosic fibers unbleached so as to leave inherent lignin present, and
applying a hydrophobic agent to at least one surface of the cellulosic fibers (such agent being applied to at least one of a surface of the cellulosic fibers and a surface of the fabric formed therefrom).
   Where a hydrophobic agent is applied, such agent may comprise at least one compound selected from the group consisting of: silica-based nanoparticles; graphene-based coatings; TiO₂ (titanium dioxide) coatings; Zinc oxide and zinc pyrithione coatings; copper oxide, copper gluconate, and copper sulfate coatings; epoxies; nanomaterial-based resins, polymers, and surfactants; Lotus effect-inspired coatings; Alkyl-based coatings; polymeric films from 1-120 μm; Self-healing hydrophobic coatings; functionalized polymers with hydrophobic side chains, such as alkyl groups; hydrophobic highly branched dendrimers; paraffins that are liquid at room temperature in water dispersible emulsions; and fatty acids.

An absorbent article may comprise an outer layer that faces outwards from the skin of the user when the article is in use, said outer layer comprising a fabric of cellulosic fibers treated with one or more hydrophobic treatments selected from the group of:
blending the cellulosic fibers with synthetic fibers,
leaving at least a portion of the cellulosic fibers unbleached so as to leave inherent lignin present, and
applying a hydrophobic agent to at least one surface of the cellulosic fibers (such agent being applied to at least one of a surface of the cellulosic fibers and a surface of the fabric formed therefrom).
   Where a hydrophobic agent is applied, such agent such agent may comprise at least one compound selected from the group consisting of: silica-based nanoparticles; graphene-based coatings; TiO₂ (titanium dioxide) coatings; Zinc oxide and zinc pyrithione coatings; copper oxide, copper gluconate, and copper sulfate coatings; epoxies; nanomaterial-based resins, polymers, and surfactants; Lotus effect-inspired coatings; Alkyl-based coatings; polymeric films from 1-120 μm; Self-healing hydrophobic coatings; functionalized polymers with hydrophobic side chains, such as alkyl groups; hydrophobic highly branched dendrimers; paraffins that are liquid at room temperature in water dispersible emulsions; and fatty acids.

Where the hydrophobic treatment includes applying a hydrophobic agent as listed above to the cellulosic fibers, such agent may be applied at the loading listed below for each type of agent:
silica-based nanoparticles may be applied at a range of 0.009-5.9% by weight (wt %),
graphene-based coatings may be applied at a range of 0.045-3.8 by weight (wt %),
TiO₂ (titanium dioxide) coatings may be applied at a range of 0.42-5.9% by weight (wt %),
Zinc oxide and zinc pyrithione coatings may be applied at a range of 0.4%-6% by weight (%),
copper oxide, copper gluconate, and copper sulfate coatings may be applied at a range of 0.005%-6% by weight (%),
epoxies may be applied at a range of 3%-15% by weight (%),
nanomaterial-based resins, polymers, and surfactants may be applied at a range of 0.5%-12% by weight (%),
Lotus effect-inspired coatings may be applied at a range of 0.36-5.9% by weight (wt %),
Alkyl-based coatings may be applied at a range of 0.008-7% by weight (wt %),
polymeric films from 1-120 μm may be applied between 5-100 gsm solids (grams per square meter),
Self-healing hydrophobic coatings may be applied at a range of 0.05%-7% by weight (%),
functionalized polymers with hydrophobic side chains, such as alkyl groups may be applied at a range of 0.36-6.2% by weight (wt %),
hydrophobic highly branched dendrimers may be applied at a range of 0.04-6.2% by weight (wt %),
paraffins that are liquid at room temperature in water dispersible emulsions may be applied at a range of 0.009-2.9% by weight (wt %), and
fatty acids may be applied at a range of 0.009-4.9% by weight (wt %).

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 is a perspective view of one example of an absorbent article, in the form of a diaper;

FIG. 2 is a cross-sectional view (not to scale) of a diaper according to a first embodiment;

FIG. 3 is a cross-sectional view (not to scale) of a diaper according to a second embodiment; and

FIG. 4 is a cross-sectional view (not to scale) of a diaper according to a third embodiment.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

As used in the description, the terms “top,” “bottom,” “above,” “below,” “over,” “under,” “above,” “beneath,” “on top,” “underneath,” “up,” “down,” “upper,” “lower,” “front,” “rear,” “back,” “forward” and “backward” refer to the objects referenced when in the orientation illustrated in the drawings, which orientation is not necessary for achieving the intended function.

A used herein, the terms “inner layer” and “top sheet” are used interchangeably to refer to the layer of the absorbent article that rests directly against the skin of the wearer when the article is in use, and which serves as the initial contact point for waste fluids and solids. The terms “outer layer”, “cover”, and “back sheet” are used interchangeably for the layer of the absorbent article that forms the external surface of the article, facing away from the skin of the wearer, when the article is in use.

As used herein the term “cellulosic materials” or “cellulosic fibers” refers to plant-derived substances primarily composed of cellulose, a natural polymer that forms the structural framework of plant cell walls. These materials encompass a variety of sources, including fibers obtained directly from plants, such as cotton, kapok, coir, flax, jute, hemp, ramie, nettle, sisal, abaca, linen, coconut, flax, bamboo, banana peels and manufactured fibers derived from natural cellulose, such as rayon, viscose, lyocell, modal, acetate, and lyocell, produced through chemical processing and cellulose extracted from the structural tissues of trees and woody plants.

As used herein the term “nonwoven” fabric or web means a web having a structure of individual fibers or threads which are interlaid, but not in an identifiable manner as in a knitted or woven fabric. Nonwoven fabrics or webs can be formed by various processes including, but not limited to spunbonding processes, spunlacing processes and bonded carded web processes.

As used herein the term “spunbond fibers” refers to small diameter fibers of mechanically and/or eductively drawn polymeric material. Spunbond fibers are generally formed by extruding molten thermoplastic material as filaments from a plurality of fine capillaries of a spinneret with the diameter of the extruded filaments then being rapidly reduced. Examples of spunbond fibers and methods of making the same are described in, by way of example only, U.S. Pat. No. 4,340,563 to Appel et al., U.S. Pat. No. 5,382,400 to Pike et al., and U.S. Pat. No. 5,795,926 to Pike et al.; the entire content of the aforesaid patents is hereby incorporated by reference herein. Spunbond fibers are generally not tacky when they are deposited onto a collecting surface and are continuous.

As used herein “spunlaced fibers” refers to those made by a process involving the cohesion and the interlacing of the elementary fibers with one another, typically by means of a plurality of jets of water under pressure passing through a moving fleece or cloth and causing the fibers to intermingle with one another. As an example, a process for the production of spunlaced nonwoven cloths has been described in U.S. Pat. Nos. 3,214,819, 3,485,706 and 3,508,308; the entire content of the aforesaid patents is incorporated by reference herein.

The word “disposable” is used in the specification to describe absorbent articles that are not intended to be laundered or otherwise restored or reused as an absorbent article. They are intended to be discarded after a single use and preferably to be disposed of in an environmentally friendly manner.

The term “silica-based nanoparticles” include various forms such as Fumed Silica, Colloidal Silica, Mesoporous Silica Nanoparticles (MSNs), Nanoporous Silica, Silica Nanospheres, Silica Nanorods, Silica Nanowires, Silica Nanotubes, Surface-modified Silica Nanoparticles, Functionalized Silica Nanoparticles, Core-shell Silica Nanoparticles, Silica Aerogels, Hollow Silica Nanoparticles, Silica-coated Nanoparticles, Organically-modified Silica Nanoparticles (ORMOSILs), and Silica Sols.

The term “Graphene-based coatings” include Graphene oxide (GO) coatings, Reduced graphene oxide (rGO) coatings, Graphene oxide/silver nanoparticle composites, Graphene oxide/polymer composites, Graphene oxide-based hybrid coatings, Graphene-based metal oxide coatings, Graphene nanoplatelet coatings, Graphene/polymer nanocomposite coatings, Graphene-based thin films, Functionalized graphene coatings, and Graphene oxide-based superhydrophobic coatings.

The term “TiO₂ (titanium dioxide) coatings” include Photocatalytic TiO₂ coatings, Hydrophobic TiO₂ coatings, Self-cleaning TiO₂ coatings, TiO₂/SiO₂ composite coatings, TiO₂ nanoparticle coatings, TiO₂ thin film coatings, TiO₂-based antimicrobial coatings, TiO₂-based anti-fog coatings, TiO₂/polymer composite coatings, TiO₂ nanowire coatings, TiO₂-coated glass, and TiO₂-based photocatalytic films.

The term “Epoxies and nanomaterial-based resins” include Epoxy nanocomposite resins, Epoxy-silica hybrid resins, Epoxy-carbon nanotube composites, Epoxy-based polyurethane nanocomposites, Epoxy-graphene nanocomposites, Epoxy-nanoclay composites, Epoxy-titania nanocomposites, Epoxy-montmorillonite composites, Epoxy-ceramic nanoparticle composites, Epoxy-nanofiber resins, Epoxy-silver nanoparticle composites.

The term “sustainable fatty acid” or “fatty acid” include stearic acid, palmitic acid, myristic acid, lauric acid, behenic acid, oleic acid, linoleic acid, arachidic acid, capric acid, caprylic acid, erucic acid, lignoceric acid, or hydroxystearic acid.

The term “Lotus effect-inspired coatings” are coatings that mimic the natural superhydrophobic properties of lotus leaves through micro- and nanostructures and include Superhydrophobic nanoparticle coatings, Microstructured silicone coatings, Hydrophobic polymer coatings with micro- and nanoscale roughness, Silica-based lotus effect coatings, Graphene oxide-based superhydrophobic coatings, Titanium dioxide-based superhydrophobic coatings, Hydrophobic micro/nanostructured surfaces, Fluoropolymer-based lotus effect coatings, Hybrid nanomaterial coatings, Self-cleaning lotus effect coatings, and Nanostructured hydrophobic films.

The term “Alkyl-based coatings” includes Alkyl silane coatings, Long-chain alkyl silane coatings, Alkylamine coatings, Alkylphosphonic acid coatings, Alkyltrichlorosilane coatings, Alkylthiol coatings, Fatty acid alkyl ester coatings, Alkyl-based polyurethane coatings, Alkyl-based epoxy coatings, Alkylated polyolefin coatings, Alkyl resin coatings, Long-chain alkyl acrylate coatings, and Alkyl-modified polymer coatings. Such coatings may create low-surface-energy layers for effective liquid resistance.

The term “self-healing coating” is defined as a type of protective layer that can automatically repair damage or degradation over time, restoring its original properties without the need for external intervention. These coatings typically contain embedded microcapsules or other mechanisms that release healing agents when the coating is scratched, cracked, or otherwise damaged. The healing agents then react to repair the damage, ensuring that the coating maintains its effectiveness, such as corrosion resistance, waterproofing, or mechanical strength. Self-healing hydrophobic coatings include, Polymer-based self-healing coatings, Polyurethane-based self-healing hydrophobic coatings, Polymers with dynamic covalent bonds, Microencapsulated healing agent coatings, Self-healing coatings with embedded microcapsules, Self-healing coatings using supramolecular chemistry, Silica-based self-healing hydrophobic coatings, Self-healing coatings with reversible cross-linking, Polyelectrolyte multilayer self-healing coatings, Graphene-based self-healing hydrophobic coatings, Self-healing coatings with encapsulated nanofillers, Polymeric networks with self-healing properties, and Self-healing hydrophobic coatings with self-repairing microstructures.

The term “functionalized polymers with hydrophobic side chains” include Poly(dimethylsiloxane) (PDMS), Poly(methyl methacrylate) (PMMA) functionalized with hydrophobic groups, Polystyrene functionalized with alkyl side chains, Polyethylene glycol (PEG) modified with hydrophobic moieties, Polyurethane with hydrophobic side chains, Polyvinyl alcohol (PVA) modified with hydrophobic groups, Polycarbonate functionalized with long-chain alkyl groups, Polypropylene functionalized with hydrophobic alkyl groups, Polytetrafluoroethylene (PTFE) derivatives with hydrophobic side groups, Poly(ethylene oxide) (PEO) with hydrophobic side chains, Polyacrylate with alkyl or fluorinated side chains, Polyamide (Nylon) functionalized with hydrophobic side groups, Polyimide with hydrophobic moieties, Polymer blends or block copolymers with hydrophobic side chains, Polyvinylchloride (PVC) with functionalized hydrophobic groups.

The phrases “personal product,” “absorbent product” and “absorbent article” are used interchangeably herein and are intended to include products or articles used to absorb waste body fluids or other body waste, and includes diapers, nappies, absorbent underpants, training pants, adult incontinence products, pet incontinence products, feminine hygiene products, wound dressings, and breast pads. It will be appreciated that the above list is not limiting and the teachings of the present disclosure can be implemented into other types of products that are intended for fluid absorption. In some cases, discussion refers to the specific example of a diaper, and many details of such examples should be understood to be applicable to other forms of absorbent articles.

Biodegradability is attributed to three main factors: “molecular weight”, “molecular number”, and “carbon content”. A lower molecular weight indicates a less polymeric product, thus easier to degrade. A higher carbon content increases the likelihood that the product will degrade without leaving residue. Additionally, products with a lower molecular number are chemically simpler and more likely to biodegrade effectively and efficiently without particulates. ASTM D5338 is a standard test method developed by the American Society for Testing and Materials (ASTM) for determining the aerobic biodegradation of materials under controlled conditions. This test evaluates how a material breaks down into carbon dioxide, water, and biomass when exposed to aerobic composting conditions. The data is reported by the percentage of carbon conversion to CO₂. In order to determine the percentage of carbon conversion, one first needs these 3 calculations to determine the starting carbon content.

A “staple fiber” is a textile fiber of a defined length that serves as a primary material in the production of nonwoven fabrics.

As noted above, renewable materials, having high cellulose content and absorbent properties are increasingly used in absorbent article components, particularly in top sheets, liners, and back sheets. However, they can retain moisture, potentially causing skin irritation if such moisture is not efficiently transferred away from the skin.

Efforts to enhance moisture removal in layers beneath the top sheet, such as fluid transfer, acquisition and distribution, backsheet, and core wrap layers, have not fully addressed the challenges of using renewable plant-based materials. While plant-derived plastics are marketed as sustainable, their synthesis involves chemical processes that transform natural materials into synthetic polymers. As these plastics degrade, they release microplastics that can become airborne, posing inhalation risks, particularly for infants, and potentially causing respiratory issues. Additionally, microplastics are biopersistent, accumulating in living organisms, making their environmental impact comparable to that of fossil fuel-based plastics, undermining their perceived sustainability.

To address these challenges, absorbent articles should align with consumer demand for plant-based cellulosic fibers in both inner and outer layers, while ensuring efficient fluid migration, retention, and leakage prevention. Eliminating petrochemical-derived and synthetic plant-based plastics in favor of fully biodegradable, non-toxic alternatives is beneficial. A solution involves developing absorbent articles made from natural materials, reducing microplastic risks, while maintaining performance. Cellulosic fibers, with their breathability and biodegradability, are ideal for top and back sheets, and their functionality can be enhanced with innovative hydrophobic treatments for eco-friendly, high-performance products.

The top sheet of a disposable diaper is traditionally manufactured using nonwoven or porous formed-film materials made from synthetic polymers like polyethylene or polypropylene. These materials are engineered for their hydrophilic properties, enabling rapid fluid transfer into the absorbent core, which contains fluff pulp and superabsorbent polymers (SAP). SAP effectively locks in moisture, while the synthetic top sheets resist rewetting due to their low moisture regain. The back sheet, typically composed of flexible polyethylene film, often incorporates a barrier layer to ensure leakproofing. This barrier may be integrated as a single component or laminated to an inner layer. Externally, the back sheet is usually a soft-touch nonwoven layer, designed to provide a comfortable feel while concealing the semi-outer film barrier. Despite their efficiency, these synthetic materials are predominantly derived from non-renewable petroleum resources.

The proliferation of disposable diapers has led to critical environmental issues stemming from the vast quantities discarded annually. Conventional diapers are largely composed of plastic-based materials such as polypropylene and polyethylene, which are neither sustainably sourced nor biodegradable. While some manufacturers have taken steps toward “green” practices—such as using recycled plastics or bio-polymers that degrade more readily—these alternatives often fall short of being truly sustainable. The processes required to produce bio-polymers, such as extrusion and enzymatic treatments, still contribute to environmental harm by emitting pollutants and generating waste. Furthermore, bio-polymers degrade into microplastics, which persist in ecosystems, disrupting wildlife and contaminating food and water supplies.

Synthetic polymers represent a monumental environmental hazard. When disposed of in landfills, they remain inert for centuries, gradually leaching harmful chemicals into the soil and groundwater. For example, an average disposable diaper—a product containing petroleum-based plastics, chemically processed wood pulp, perfumes, and bleaching byproducts like dioxins—can take up to 500 years to decompose. During this prolonged degradation process, the materials release toxic substances that accumulate in the environment, posing long-term risks to both human health and ecosystems.

To address these challenges, there is a growing demand for absorbent articles made from sustainable, plant-based materials derived from cellulosic structures. These natural materials offer a promising alternative, providing absorbency, reduced rewetting, effective leak prevention, and a soft, gentle feel in both the inner and outer layers of the product. These sustainable designs aim to perform as well as or better than conventional plastic-based diapers while reducing their environmental footprint. Even if they do not fully meet current biodegradability testing standards, such plant-based absorbent structures represent a significant step toward mitigating the environmental impact of disposable hygiene products.

Synthetic bio-polymers, which are still problematic but are often marketed as eco-friendly alternatives to traditional plastics, carry their own set of environmental drawbacks. Their production typically involves resource-intensive processes such as polymerization, chemical treatments, and extrusion, all of which contribute to greenhouse gas emissions and the release of industrial pollutants. Furthermore, many bio-polymers are designed to degrade only under specific industrial composting conditions, which are not widely available. When disposed of in conventional waste streams, they often degrade into microplastics, perpetuating the same environmental challenges as traditional plastics. These microplastics infiltrate soils, waterways, and food chains, causing irreversible ecological damage. The energy and resources required to produce synthetic bio-polymers can outweigh their perceived benefits, rendering them an inadequate solution for achieving true sustainability.

A shift to natural, cellulosic materials can address these shortcomings, offering a renewable, biodegradable alternative that minimizes harm throughout the product's lifecycle.

Many absorbent products are now incorporating natural fibers such as cotton or bamboo, often blended with other materials. These natural fibers, being cellulosic, are typically used across various layers but are most commonly utilized in the top sheet. Unlike synthetic materials, natural fibers tend to absorb and retain liquid instead of efficiently transferring it to the absorbent core, where moisture can be locked away from the skin. As a result, users of absorbent products with natural fiber top sheets may experience increased skin wetness. Rewet testing data confirms that these natural fiber-based top sheets have a higher tendency for liquid reabsorption compared to their synthetic counterparts. To overcome this challenge, top sheets made from natural fibers need to be engineered to emulate the moisture transfer and rewet-resistant properties of synthetic materials. The focus is on creating a top sheet with a high strike-through rate and low rewet values to enhance skin dryness and comfort.

Similarly, absorbent products such as feminine hygiene pads and incontinence solutions share structural similarities with disposable diapers, including the presence of a top sheet and a moisture-locking absorbent core. However, none of the currently available products in these categories feature advanced top sheets made from natural fibers that effectively minimize moisture retention while maintaining skin dryness. Developing such innovative natural fiber-based top sheets presents a significant opportunity to improve product performance in these markets.

Accordingly, the present disclosure describes sustainable absorbent structures made from cellulosic materials, addressing and resolving the limitations of existing designs to create hydrophobic, antimicrobial, and sustainable top sheets and back sheets. This redefined use of cellulosic materials emphasizes their role as sustainable, versatile components, further enhanced by advanced hydrophobic treatments to meet the performance and environmental demands of modern absorbent products.

In one embodiment, the absorbent article features a hydrophobic inner layer or “top sheet” made from cellulosic nonwoven fabric. This top sheet enhances the article's performance by directing liquids to the core rather than retaining them on the surface or absorbing them into the cellulosic structure, as seen in conventional cellulosic-based top sheets. The hydrophobic top sheet may offers excellent low rewet performance, leakage prevention, and a soft, supple texture. Additionally, it may replicate or surpass the performance of standard plastic-based absorbent articles while remaining sustainable, biodegradable, and free from petroleum-based chemicals. It may also incorporate a cellulosic-comprising ADL layer, cellulosic comprising core wrap, and a cellulosic comprising back sheet.

The top sheet may comprise natural materials derived from cellulosic fibers that also contains a plurality of pores and a hydrophobic property that does not interfere with the sustainability of the top sheet itself, but enhances its rewet characteristics so that it performs more like that of standard plastic unsustainable top sheets while adding comfort and breathability for the wearer.

In one embodiment, the top sheet is comprised entirely or substantially of sustainable materials that in diaper form has a rewet of less than 40 grams and a strike through faster than 8 seconds using the INDA single strike through, re-wet, and multiple strike through standardized testing methods. And such absorbent articles, including diapers also have a soft hand feel on its inward facing surface.

The top sheet for use in disposable absorbent articles may comprise substantially cellulosic fibers and be hydrophobic. Such top sheet also may comprise a plurality of pores.

In a further embodiment, the top sheet for use on disposable absorbent articles includes a non-woven inner layer comprising cellulosic materials that is either unbleached, to maintain the hydrophobic properties offered by intact lignin, and/or treated to include at least one compound selected from the group consisting of Silica-based nanoparticles, Graphene-based coatings, TiO₂ (titanium dioxide) coatings, Zinc oxide and zinc pyrithione coatings, copper oxide, copper gluconate, or copper sulfate coatings, epoxies and nanomaterial-based resins, polymers and surfactants for hydrophobic soil applications adapted for cellulosic substrates, Lotus effect-inspired coatings that mimic the natural superhydrophobic properties of lotus leaves through micro- and nanostructures, Alkyl-based coatings, polymeric films, Self-healing hydrophobic coatings, carbon nanotubes and fullerenes, functionalized polymers with hydrophobic side chains, specifically synthesized hydrophobic highly branched dendrimers, paraffins that are liquid at room temperature in water dispersible emulsions, or sustainable fatty acids.

In certain cases, the treatment is applied to the inner layer at 0.009% to 5.35% solids by weight add on. In certain embodiments treatment is applied at about 2.75% by weight add on. In certain embodiments treatment is applied at about 0.28% by weight add on. In some embodiments, the hydrophobic treatment is applied only to the top surface of the inner layer. In certain other embodiments, the treatment is applied to the bottom and top surface of the inner layer. In some embodiments, treatment is applied to staple fibers used to form the top sheet prior to forming the fibers into the non-woven fabric.

In certain embodiments, there is no treatment applied to the inner layer or outer layer rather the respective layer or layers are comprised of cellulosic fibers blended with synthetic fibers such as polyethylene, polypropylene, polyester, Polylactic Acid (PLA), Polyhydroxyalkanoates (PHA), Cellulose Acetate, Starch-Based Plastics, Polybutylene Succinate (PBS), Polytrimethylene Terephthalate (PTT), Polyethylene Furanoate (PEF), Bio-Polyethylene (Bio-PE), Bio-Polypropylene (Bio-PP), Bio-Polyamide (Bio-PA), Lignin-Based Plastics, Natural Rubber-Based Bioplastics, Soy Protein-Based Plastics, Algae-Based Plastics, Chitin or Chitosan-Based Plastics, or Hemicellulose-Based Plastics or other thermoplastic polymer matrix materials. The layer comprised of cellulosic fibers as blended with not more than 90% synthetic fibers, not more than 75% synthetic fibers, not more than 50% synthetic fibers, or not more than 25% synthetic fibers, or not more than 5% synthetic fibers by weight in the layer itself. In certain embodiments, the total carbon content of the resulting absorbent article is higher than 50 (g/kg) or 50% expressed as a percent of carbon to the total mass, the molecular number is lower than 320 (mol) moles and the molecular weight is less than 900 (g/mol) grams per mole.

In certain embodiments, there is no treatment applied to the inner layer or outer layer, rather one or both are comprised of cellulosic fibers blended with other unbleached cellulosic fibers keeping the lignin intact on the cellulosic structure thereby imparting a natural hydrophobic property to the fibers themselves. The cellulosic fibers can be 100% unbleached or blended with not more than 90% bleached fibers, not more than 75% bleached fibers, not more than 50% bleached fibers, or not more than 25% bleached fibers or not more than 5% bleached fibers by weight in the layer itself.

In certain embodiments, the inner layer of the absorbent article is treated with a sustainable fatty acid. In some of those embodiments, the treatment is applied to a top surface of the inner layer (the top surface being the surface that rests against the skin of the wearer when the article is in use). In some embodiments, the treatment is applied to a top and a bottom surface of the inner layer.

In certain embodiments of the absorbent article, the inner layer includes a plurality of pores.

Absorbent articles so comprised may, for example, be formed to provide a diaper, a nappy, absorbent underpants, training pants, adult incontinence products, pet incontinence products, feminine hygiene products, wound dressings, or breast pads.

A sustainable diaper or other absorbent article can be formed with a non-woven inner layer (“top sheet”) comprising cotton treated with a hydrophobic agent selected from the group consisting of Silica-based nanoparticles, Graphene-based coatings, TiO₂ (titanium dioxide) coatings, Zinc oxide and zinc pyrithione coatings, copper oxide, coper gluconate, or copper sulfate coatings, epoxies and nanomaterial-based resins, polymers and surfactants for hydrophobic soil applications adapted for cellulosic substrates, Lotus effect-inspired coatings that mimic the natural superhydrophobic properties of lotus leaves through micro- and nanostructures, Alkyl-based coatings that are long-chain alkyl silanes or similar compounds create low-surface-energy layers for effective liquid resistance, polymeric films, Self-healing hydrophobic coatings, carbon nanotubes and fullerenes, functionalized polymers with hydrophobic side chains, specifically synthesized hydrophobic highly branched dendrimers, paraffins that are liquid at room temperature in water dispersible emulsions, or sustainable fatty acids and combinations thereof, an outer layer comprising cotton treated with a hydrophobic agent selected from the same group of agents (either the same or different agent(s) as applied to the inner layer), and a core positioned between the inner layer and the outer layer.

In one embodiment, the sustainable absorbent structure includes a hydrophobic outer layer (“back sheet”) made from cellulosic nonwoven fabric. This back sheet may improve the article's performance by preventing liquid from passing through to the exterior if the core becomes saturated, while also blocking external liquids from penetrating the article and reaching the core. The hydrophobic cellulosic back sheet may provide exceptional liquid repellency, zero wicking capacity, leakage prevention, and a soft, supple feel.

In one embodiment, the back sheet is comprised entirely or substantially of sustainable materials that the flat fabric, before converting into diaper form, has a vertical wick of less than 70 mm using AATCC TM197-2022 standardized testing methods and a water repellency rating of 75 or higher using AATCC TM22 standardized testing methods. When incorporated into an absorbent article, including diapers, such back sheet may have a soft hand feel on its outward-facing surface.

In certain cases, the treatment is applied to the outer layer at 0.05% to 7.75% solids by weight add on. In certain embodiments, treatment is applied at about 2.25% by weight add on. In some embodiments, the hydrophobic treatment is applied only to the top surface of the outer layer (the top surface being the layer facing towards the inner layer of the absorbent article when constructed). In certain other embodiments, the treatment is applied to the bottom and top surfaces of the outer layer.

In one embodiment, the resulting absorbent article biodegrades in less than 28 months under ASTMD 5338 standardized test method.

In one embodiment, the staple fibers used to form the outer layer and/or the outer layer are treated prior to fabric formation at a treatment level of less than 5% solids by weight add on.

In certain embodiments, the outer layer of the absorbent article is treated with a sustainable fatty acid. In some of those embodiments, the treatment is applied to a top surface of the outer layer (the top surface being the layer facing towards the inner layer of the absorbent article when constructed). In some embodiments, the treatment is applied to a top and a bottom surface of the outer layer.

In some cases, the diaper or other absorbent article has a treatment consisting of linoleic acid applied to both the inner layer and outer layer. In certain of those cases, the linoleic acid treatment is applied to the inner layer at 0.009% to 2.35% solids by weight add on. In certain embodiments the linoleic acid is applied at about 1.75% by weight add on. In some embodiments, the hydrophobic linoleic acid treatment is applied only to the top surface (the surface facing inwards against the skin of the user when the article is worn) of the inner layer and in certain cases, the linoleic acid treatment is also applied to the outer layer at 0.009% to 5.75% solids by weight add on. In certain embodiments treatment is applied at about 1.25% by weight add on. In some embodiments, the hydrophobic linoleic acid treatment is applied only to the top surface (the surface facing towards the inner layer) of the outer layer.

In certain embodiments, the diaper has a rewet of less than 40 grams and a strike through faster than 8 seconds using the INDA single strike through, re-wet, and multiple strike through standardized testing methods. Typically, the strike through and re-wet properties have an inverse relationship. When the strike through is fast, the re-wet is higher and vice versa.

Table 1 that follows presents multiple strike through data generated using INDA standardized testing methods and compares the linoleic acid treated top sheet in a diaper to commercially available plastic diapers as well as a 100% cotton untreated top sheet. Performance is considered acceptable when the Multiple Strike through is less than 6 seconds.

TABLE 1

| Diaper ID | MST 1 | MST 2 | MST 3 | MST 4 | MST 5 |
|---|---|---|---|---|---|
| Linoleic acid treated | 1.07 | 2.35 | 3.17 | 3.45 | 3.86 |
| Top Sheet Diaper | | | | | |
| Pampers ^{®} Swaddlers | 2.10 | 4.11 | 5.16 | 5.61 | 4.33 |
| Luvs ® | 1.27 | 1.80 | 1.83 | 2.67 | 1.84 |
| Huggies ® Snug | 1.31 | 1.72 | 2.31 | 2.87 | 3.00 |
| and Dry | | | | | |
| Untreated 100% cotton | 0.34 | 1.25 | 1.77 | 2.34 | 2.68 |

Table 2 that follows presents Single Strike Through data that was generated using INDA standardized testing methods and compares the linoleic acid treated top sheet in a diaper to commercially available plastic diapers as well as a 100% cotton untreated top sheet. Performance is considered acceptable when the Single Strike through is less than 6 seconds.

TABLE 2

| Diaper ID | SST, seconds |
|---|---|
| Linoleic acid treated Top Sheet Diaper | 1.14 |
| Pampers ® Swaddlers | 2.14 |
| Luvs ® | 1.63 |
| Huggies ® Snug and Dry | 1.40 |
| Untreated 100% cotton | 0.66 |

Table 3 that follows presents re-wet data that was generated using INDA standardized testing methods with sterile solution of sodium chloride (salt) in water at a concentration of 0.9% NaCl (standardized saline concentration) and compares the linoleic acid treated top sheet in a diaper to commercially available plastic diapers as well as a 100% cotton untreated top sheet. Performance is considered acceptable when the re-wet is less than 0.25 grams.

TABLE 3

| Diaper ID | Re Wet, grams |
|---|---|
| Linoleic acid treated Top Sheet Diaper | 0.09373 |
| Pampers ® Swaddlers | 0.09503 |
| Luvs ® | 0.07290 |
| Huggies ® Snug and Dry | 0.08687 |
| Untreated 100% cotton | 0.69870 |

Table 4 that follows presents molecular weight, molecular number and carbon content (as measured by ASTM D5338 test) of the linoleic acid treated top sheet and linoleic acid treated back sheet in a diaper as compared to the molecular weight, molecular number and carbon content of a commercially available plastic diaper, in this instance a Pampers® diaper was used. The measurements in the chart are on the entire absorbent article (complete diaper), not the individual layers.

TABLE 4

| | Preferable | Pampers ® |
|---|---|---|
| | Embodiment Diaper | Swaddlers |
| Molecular Weight (g/mol) | 296 | 790 |
| Molecular Number (mol) | 168 | 301 |
| Carbon Content (g/kg) | 91.3 | 72.6 |

Table 5 that follows presents AATCC TM197-2022 vertical wicking data and compares two different embodiment treated back sheets to a commercially available petroleum based plastic diaper back sheet as well as a 100% cotton untreated back sheet.

TABLE 5

| | Distance Wicked | Distance Wicked | Distance Wicked |
|---|---|---|---|
| AATCC TM197-2022 | at 2 min (120 s) | at 10 min (600 s) | at 30 min (1800 s) |
| Untreated Cotton nonwoven | 77.0 mm | 141.0 mm | 168.0 mm |
| Copper Sulfate treated cellulosic nonwoven | 34.0 mm | 48.0 mm | 67.0 mm |
| capric acid treated cellulosic nonwoven | 0.0 mm | 0.0 mm | 0.0 mm |
| Polypropylene nonwoven | 75.0 mm | 125.0 mm | 187.0 mm |

Table 6 that follows presents AATCC TM22 data that measures the resistance of fabrics to wetting by water. Ratings typically range from 0 to 100, with 100 representing the most water repellent fabric and lower numbers indicating increasing levels of wetting. Performance is considered water repellant and acceptable when the rating is above 70.

TABLE 6

| AATCC TM22 | Rating |
|---|---|
| Untreated Cotton nonwoven | 0.0 |
| Copper Sulfate treated cellulosic nonwoven | 70.0 |
| capric acid treated cellulosic nonwoven | 100.0 |
| Polypropylene nonwoven | 100.0 |

Table 7 that follows presents ASTM F1249 standard water vapor transmission rate of films. The higher the MVTR (moisture vapor transfer rating) the more breathable the material. This data is based on back sheet and semi-outer layer film materials tested together as one combined outer layer unit.

TABLE 7

| Diaper ID | MVTR grams/m2/day |
|---|---|
| capric acid treated Back Sheet | 625 |
| Pampers ® Swaddlers | 249 |
| Luvs ® | 462 |
| Huggies ® Snug and Dry | 204 |

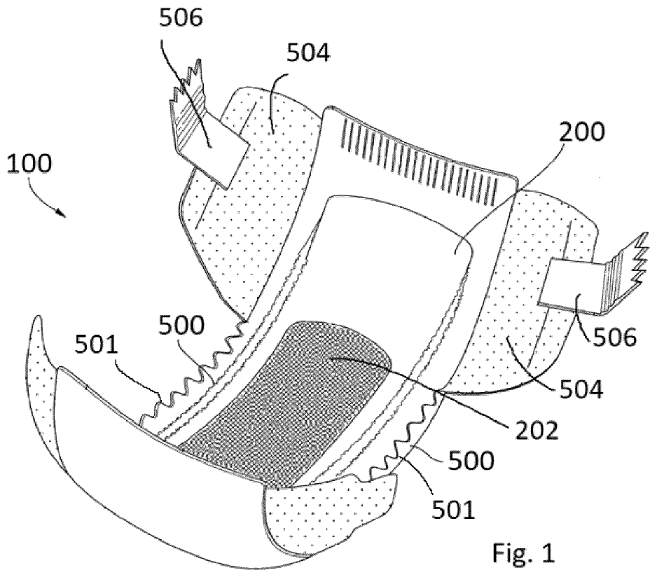

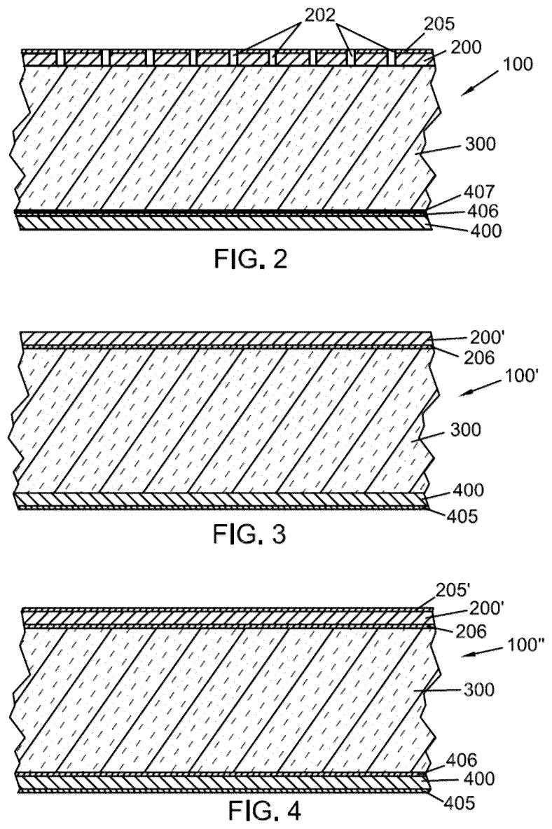

FIGS. 1 and 2 illustrate one example of an absorbent article (personal product) 100 according to one embodiment and, in particular, the absorbent article 100 illustrated is in the form of a disposable diaper for an infant (baby). It will be understood that the illustrated diaper 100 is only one example and the discussion of features could be applied to a toddler training pant/diaper, other similar absorbent products, as well as other absorbent products that may or may not be mentioned herein. FIG. 1 is a perspective view of an embodiment of a diaper 100, while FIG. 2 is a cross-sectional view of a portion of the diaper 100 showing the layered structure.

The diaper 100 is formed generally of an inner layer (top sheet) 200, an inner acquisition distribution layer and/or absorbent core 300, a film layer 407 and an outer shell or cover (back sheet) 400, as shown.

In some instances, there are also side panels, otherwise known as leg cuffs 500 (see FIG. 1), which are designed to effectively prevent leakage at the sides of the diaper 100, thereby keeping the skin dry and protected. In some embodiment, the leg cuffs 500 can be constructed as a set of inner standing leg cuffs or gathers 501, as well as the (outer) leg cuffs 500. As shown in FIG. 1, the leg cuffs 500 or gathers 501 (not shown) are generally formed in the central portion of the diaper 100. In the FIG. 1 diaper, each leg cuff 500 may be formed of a nonwoven hydrophobically treated cellulosic fiber structure with a spandex inlay to provide the desired elasticity. When the diaper 100 includes a defined elastic waistband, it too can be formed of a nonwoven material that contains cellulosic fibers with a spandex inlay to provide elasticity thereto.

It will be appreciated that the elastic structure can be formed of other materials, such as spandex and other synthetics. As mentioned herein, the elastic structures are used in leg cuffs and also, they can be used in lateral side panels and in tape constructions. For that matter, many gasketing cuffs use spandex to provide a seal with the infant's legs.

In some embodiments, in order to keep the diaper 100 attached, and substantially sealed against the wearer's body, flaps 504 with fasteners 506 are included.

The inner layer 200 plays an important role in the construction of the diaper since the inner layer 200 is the layer that is next to the most sensitive areas of the baby's body, sometimes for hours at a time. The quality and makeup of the inner layer 200 layer also determines the level of absorbency, rewet, strikethrough and comfort (softness) for the baby.

In the embodiment illustrated, the inner layer 200 is formed from cellulosic fibers (e.g., 100% organic cotton or coconut). More specifically, the inner layer 200 comprises a non-woven fabric (material) that is formed from cellulosic fibers. Any number of different cellulosic fibers can be used to form the non-woven inner layer 200 so long as they are suitable for the intended use described herein. Examples of cellulosic fibers include but are not limited to limited to: cotton, linen, coconut, flax, bamboo, viscose, etc. Cotton is the most widely used natural fiber and is absorbent, easy to care for, and comfortable for babies. Cotton is almost pure cellulose and is both soft to the touch and has advantageous breathability. As with many materials, cotton is available and marketed in an organic form which is generally understood to mean that the cotton is grown without the use of any synthetic agricultural chemicals, such as fertilizers or pesticides. It will also be appreciated that the inner layer 200 can be formed as a blend of two or more cellulosic materials.

It will also be appreciated that the inner layer 200 can be formed as a blend of any of the above-mentioned natural materials and synthetic plant-derived plastics such as Polylactic Acid (PLA), Polyhydroxyalkanoates (PHA), Cellulose Acetate, Starch-Based Plastics, et al. It will also be appreciated that the inner layer 200 can be formed as a blend of any of the above-mentioned natural materials and thermoplastic polymers such as polyethylene, polypropylene, polyester, et al.

Cotton-based personal products/absorbent articles may be constructed of layers formed using spunbond or spunlaced fibers.

The inner layer 200 can be either hydrophobically treated on one side and hydrophilically treated on the other or hydrophobically treated on just one or both sides. More particularly, the non-woven material forming the inner layer 200 can undergo a surface treatment using a treatment composition which either imparts hydrophilic properties or hydrophobic properties. Any number of different surface treatments can be used to impart increased hydrophilicity to the nonwoven fabric. For example, chemicals, such as surfactants can increase the hydrophilic nature of the material and can make a fabric that is naturally hydrophobic into one that is hydrophilic. With respect to fibers, natural fibers tend to be hydrophilic, especially when natural oils have been removed from them. The surface treatment can be performed using any number of suitable techniques and more specifically, conventional treatments involve steps such as dipping the nonwoven in a treatment bath, coating or spraying the nonwoven with the treatment composition, and printing the nonwoven with the treatment composition or treating some or all of the staple fibers themselves prior to fabric formation by means of dipping in a treatment bath, coating or spraying some or all of the staple fibers with the treatment composition.

The surface treatment of the top sheet layer can be configured to increase the hydrophobic nature of the inner layer 200 by decreasing the surface tension of the inner layer 200, and allowing the fluid to be fully repellent when presented on the top sheet, if the fluid is introduced to the top sheet independently of the full diaper structure.

As shown in FIG. 1, and which can be applied in other embodiments, the inner layer 200 comprises a plurality of openings or pores 202, such as those provided in a gauze-like material. These openings (pores 202) readily allow for the passage of fluid into the inner acquisition/distribution layer and/or absorbent core 300 (FIG. 2) so that fluid is not retained on the surface of the nonwoven inner layer 200 that touches the skin.

The nonwoven inner layer 200 can be formed by any number of suitable processes including, but not limited to, hydroentanglement, carding, air-laying, thermal treatment, or other suitable process. Pores, a dot matrix, or perforations can be created during the nonwoven formation process or can be applied to the nonwoven after formation. Any number of means can be employed to create these openings (e.g., pores 200) in a pre-formed nonwoven, including but not limited to air jet formation, laser formation, during extrusion or casting, or by passing a sheet through a roller mill wherein the rollers have microspikes suitable for forming pores on the sheet. Additionally, a porous sheet can be formed by incorporating microparticles of a water-soluble substance and passing the sheet through a water bath after its formation. The water dissolves the microparticles so as to produce pores. In particular embodiments, the pores are structurally built during the cellulosic containing sheet formation.

The openings or pores 202 in the nonwoven inner layer 200 may be any shape that allows the passage of fluid, such as oval or round. The openings are typically between about 0.07 cm and about 0.40 cm in diameter. The pores 202 of the nonwoven inner layer 200 may be 0.18 cm to 0.23 cm in diameter.

In addition, both the plurality of pores 202 and the hydrophobic nonwoven top sheet 200 create a system of which increases the rewet performance and maintains the fluid near the core 300 of the diaper preventing wetness from reaching back up through the diaper to the skin. The surface treatment of the top sheet 200 is thus configured to increase the hydrophobic nature of the inner layer 200 by decreasing the surface tension of the inner layer 200, allowing the fluid to repel. This tension is at such a level that stand alone the top sheet with the plurality of pores will hold a bead of water and will not allow the water to go through the pores on its own merit without a superior fast wetting surfactant located on the inner surface of the top sheet itself (FIG. 3. 206) or below the top sheet structure in either in the ADL layer, core wrap or core itself.

FIG. 2 shows a surface treatment 205 applied to a top surface of the nonwoven inner layer 200, i.e., the surface in contact with skin. FIGS. 3 and 4 present alternative embodiments (100′, 100″) that both show a surface treatment 206 applied to a bottom surface of the nonwoven inner layers 200′, i.e. the surface closer to the respective cores 300. The FIG. 4 embodiment 100″ illustrates an example where the surface treatment 205′, 206 is applied to both the top and bottom surfaces of the inner layer 200′ (this could be the same treatment for both surfaces 205′, 206 or a different treatment for each).

It will also be understood that the surface treatment applied to the inner layer 200 can be configured as a finish that imparts hydrophobic properties on either one side and the other side is left in its natural state, as a finish that imparts hydrophobic properties on one side and as a finish that imparts hydrophilic properties the other side, or hydrophobic on both sides of the inner layer.

It will also be understood that, if the staple fibers are treated themselves prior to fabric formation, the fibers and treatment will extend throughout the top sheet 200. Should only parts of the fibers be treated and comingled with untreated fibers, cellulosic or otherwise, prior to fabric formation, it is understood that the treated fibers will be interspersed in the nonwoven web of the inner layer 200 itself and not limited to one surface or the other.

The outer layer 400 plays an important role in the construction of the diaper since the outer layer 400 is the layer that is the outer-most exterior portion or outer shell of the absorbent article. The quality and makeup of the outer layer 400 layer also determines the level of leak resistance of the article itself and comfort (softness) for the caregiver.

In one example of the present absorbent article, the outer layer 400 is formed from cellulosic fibers (e.g., 100% organic cotton or coconut). More specifically, the outer layer 400 may comprise a non-woven fabric (material) that is formed from cellulosic fibers. Any number of different cellulosic fibers can be used to form the non-woven outer layer 400 so long as they are suitable for the intended use described herein. Examples of cellulosic fibers include but are not limited to limited to: cotton, linen, coconut, flax, bamboo, viscose, etc. Cotton is the most widely used natural fiber and is absorbent, easy to care for, and comfortable for babies. Cotton is almost pure cellulose and is both soft to the touch and advantageously has breathability. It will also be appreciated that the outer layer 400 can be formed as a blend of two or more cellulosic materials.

As shown in FIG. 2, the outer layer 400 may be laminated with a semi-outer film layer 407. Externally, the back sheet 400 may be a soft-touch nonwoven layer, designed to provide a comfortable feel while concealing the semi-outer film layer 407 that serves as a barrier. The outer layer 400 along with the semi-outer film layer 407 may in combination have an MVTR rating of higher than 1000.

It will also be appreciated that the outer layer 400 can be formed as a blend of any of the above-mentioned natural materials and synthetic plant-derived plastics such as Polylactic Acid (PLA), Polyhydroxyalkanoates (PHA), Cellulose Acetate, Starch-Based Plastics, et al.

It will also be appreciated that the outer layer 400 can be formed as a blend of any of the above-mentioned natural materials and thermoplastic polymers such as polyethylene, polypropylene, polyester, et al.

Cotton-based personal products/absorbent articles may be constructed of layers formed using spunbond or spunlaced fibers.

The outer layer 400 can be either hydrophobically treated on one side and hydrophilically treated on the other or hydrophobically treated on just one or both sides. More particularly, the non-woven material forming the outer layer 400 can undergo a surface treatment using a treatment composition which either imparts hydrophilic properties or hydrophobic properties. Any number of different surface treatments can be used to impart increased hydrophilicity to the nonwoven fabric. For example, chemicals, such as surfactants, wetting agents and rewetting agents can increase the hydrophilic nature of the material and can make a fabric that is naturally hydrophobic into one that is hydrophilic. With respect to fibers, natural fibers tend to be hydrophilic, especially when natural oils have been removed from them. The surface treatment can be performed using any number of suitable techniques and more specifically, conventional treatments involve steps such as dipping the nonwoven in a treatment bath, coating or spraying the nonwoven with the treatment composition, and printing the nonwoven with the treatment composition or treating some or all of the staple fibers themselves prior to fabric formation by means of dipping in a treatment bath, coating or spraying some or all of the staple fibers with the treatment composition.

The surface treatment of the back sheet layer is configured to increase the hydrophobic nature of the nonwoven outer layer 400 by decreasing the surface tension of the nonwoven allowing the fluid to be fully repellent when presented on the back sheet, if the fluid is introduced to the back sheet independently of the full diaper structure.

The nonwoven outer layer 400 can be formed by any number of suitable processes including, but not limited to, hydroentanglement, carding, air-laying, thermal treatment, or other suitable process.

FIG. 2 shows a surface treatment 406 applied to a top surface of the nonwoven outer layer 400, i.e., i.e. the surface closer to the respective cores 300. FIGS. 3 and 4 present alternative embodiments (100′, 100″) that both show surface treatment 405 applied to a bottom surface of the nonwoven outer layers 400, the outermost surface in contact with the caregiver. The FIG. 4 embodiment 100″ includes that the surface treatment 405, 406 is applied to both the top and bottom surfaces of the outer layer 400. While these embodiments (100′, 100″) are shown without a semi-outer film layer 407, such could be employed in these embodiments. Similarly, alternative combinations of treatments of the inner layer 200 and the outer layer 400 than those illustrated could be employed.

It will also be understood that the surface treatment applied to the outer layer 400 can be configured as a finish that imparts hydrophobic properties on either one side and the other side is left in its natural state, as a finish that imparts hydrophobic properties on one side and as a finish that imparts hydrophilic properties the other side, or hydrophobic on both sides of the outer layer.

It will also be understood should the staple fibers be treated themselves prior to fabric formation, the fibers will be throughout the back sheet. Should only parts of the fibers be treated and comingled with untreated fibers, cellulosic or otherwise, prior to fabric formation, it is understood that the treated fibers will be interspersed in the nonwoven web itself and not limited to one surface or the other.

In some embodiments, the treatment may comprise Starburst® PAMAM dendrimer wax dispersion, Nujol® a highly purified mineral oil also commonly referred to as a liquid paraffin-based emulsion, Hempaprime CUI 275 a fast-drying, alkylamine-cured epoxy coating. One form may be provided as stearic acid from by Sigma-Aldrich. In certain embodiments, the treatment may comprise palmitic acid from TCI America.

In certain embodiments, the hydrophobic treatment comprises wax emulsions or a fatty acid emulsion. Emulsions that may be used in the article are available in liquid form and can be applied to nonwoven materials by a number of known application techniques. An advantage of emulsion-based treatments is the mitigated environmental impact as opposed to fluorocarbons.

In one embodiment, the treatment is applied to the cellulosic fibers themselves, prior to fabric formation at 0.009% to 15% solids. In some embodiments, 0.015% to 12.05% solids of the treatment are applied. In certain of those embodiments, about 0.25% by weight of the treatment is applied. In certain of those embodiments, about 10% by weight of the treatment is applied. In particular embodiments, 0.15% to 3.35% by weight of the treatment is applied to fabric. In many cases, at least 0.009% add on by weight of the hydrophobic treatment is applied to the layer intending to impart hydrophobicity on. It is believed that the use of lower levels of the stated ranges of such ingredients allow the treated fabrics to retain hydrophilic characteristics, as opposed to the water resistant and water repellant properties imparted by higher concentrations of such chemicals to fabrics.

As used herein, the terms “percent”, “%,” “solids percent” and “wt %” all mean the percentage by weight of the indicated component or ingredient within the product or composition in which it is present, without dilution after drying, unless otherwise indicated by the context in which the term is used. When the treatment is applied to a fabric, the percent”, “%,” “solids percent” and “wt %” refers to the amount applied to the fabric upon drying unless otherwise stated. Treatment solutions and suspensions comprise various percentages by weight of the treatment compositions in water unless another solvent/diluent is indicated.

An external hydrophobic agent is applied to the surface of the nonwoven web. An internal hydrophobic agent is blended with the material used to form the nonwoven web, and later migrates to the surface after the nonwoven web is formed. External and internal hydrophobic agents can be characterized in terms of their durability and wettability similar to hydrophilic agents as described above.

As mentioned above, a surface treatment can be applied to the inner layer 200 to impart hydrophobicity as well as the outer layer 400 to also impart hydrophobicity.

The inner layer 200 is thus a soft, comfortable hydrophobic layer formed of a cellulosic that resists moisture regain and does not hold liquid next to the surface of the absorbent article but rather transfers it to the core where it is held.

The outer layer 400 is thus a soft, comfortable hydrophobic layer formed of a cellulosic that resists moisture regain and does not hold liquid next to the surface of the absorbent article but rather prevents leakage from the core of the article and prevents penetration of external liquids from entering the article.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An absorbent article comprising:
a layer that comprises a fabric of cellulosic fibers treated with at least one hydrophobic treatment selected from the group of:
blending with synthetic fibers,
leaving at least a portion of said cellulosic fibers unbleached so as to leave inherent lignin present, and
applying a hydrophobic agent to at least one surface of said cellulosic fibers, such agent being applied to at least one of a surface of said cellulosic fibers and a surface of said fabric, and applied at a solid loading percent of 15% or less.

2. The absorbent article of claim 1 wherein said at least one hydrophobic treatment includes applying to at least one side of said cellulosic fibers at least one compound selected from the group consisting of: silica-based nanoparticles; graphene-based coatings; TiO₂ (titanium dioxide) coatings; zinc oxide and zinc pyrithione coatings; copper oxide, copper gluconate, and copper sulfate coatings; epoxies; nanomaterial-based resins, polymers, and surfactants; lotus effect-inspired coatings; alkyl-based coatings; polymeric films; self-healing hydrophobic coatings; functionalized polymers with hydrophobic side chains; hydrophobic highly branched dendrimers; paraffins that are liquid at room temperature in water dispersible emulsions; and fatty acids.

3. The absorbent article of claim 1 wherein the absorbent article is selected from the group consisting of: diapers, nappies, absorbent underpants, training pants, adult incontinence products, pet incontinence products, feminine hygiene products, wound dressings, and breast pads.

4. The absorbent article of claim 1, wherein said hydrophobic treatment comprises at least one treatment selected from the group of:
applying, at least one of a fatty acid and a fatty acid emulsion to at least one surface of said cellulosic fibers;
comprises applying at least one of a paraffin wax and a paraffin wax emulsion to at least one surface of said cellulosic fibers; and
applying a hydrophobic agent to the cellulosic fibers before fabric formation at less than 5% by weight add on.

5. The absorbent article of claim 1 further comprising a second layer and a core interposed between said layers, and wherein said layers and said core are free from oil based microplastic-emitting materials and petrochemical derivatives.

6. The absorbent article of claim 1, wherein the article as a whole has a total carbon content greater than 50, a molecular number less than 320 and molecular weight less than 900.

7. The absorbent article of claim 1, having a semi-outer layer wherein the semi-outer layer has an MVTR rating of at least 600 g/m²/day.

8. The absorbent article of claim 1 wherein said layer is an inner layer that rests against the skin of the user when the article is in use.

9. The absorbent article of claim 8, wherein said hydrophobic treatment comprises applying a hydrophobic agent to only a top surface of the inner layer, said top surface facing towards the skin of the wearer when the article is in use.

10. The absorbent article of claim 8, wherein said hydrophobic treatment comprises applying a hydrophobic agent to said inner layer at 0.009% to 2.75% by weight add on.

11. The absorbent article of claim 8 having a strike-through rate of less than 8 seconds and a rewet value of less than 40 grams as per INDA standardized testing, when tested using a sterile solution of sodium chloride (salt) in water at a concentration of 0.9% NaCl (standardized saline concentration).

12. The absorbent article of claim 8, further comprising at least one additional layer selected from the group of:
a hydrophobic nonwoven outer layer comprising cellulosic nonwoven fibers combined with a semi-outer film layer,
said outer layer and said film layer, in combination with said inner layer, providing the article with a strike-through rate of less than 8 seconds and a rewet value of less than 40 grams as per INDA standardized testing with sterile solution of sodium chloride (salt) in water at a concentration of 0.9% NaCl (standardized saline concentration);
an ADL layer that comprises cellulosic fibers; and
an absorbent core layer that is encapsulated in cellulosic fibers.

13. The absorbent article of claim 8, wherein at least one surface of said inner layer comprises a plurality of pores.

14. The absorbent article of claim 1 wherein said layer is an outer layer that faces outwards from the skin of the user when the article is in use and wherein said hydrophobic treatment comprises applying a hydrophobic agent to said outer layer at 0.05% to 5.25% by weight add on.

15. The absorbent article of claim 14, wherein said layer is an outer layer that faces outwards from the skin of the user when the article is in use and has an MVTR rating of at least 600 g/m²/day.

16. The absorbent article of claim 14, wherein said layer is an outer layer that faces outwards from the skin of the user when the article is in use and is configured to achieve a vertical wicking capability of less than 70 mm using AATCC TM197-2022 testing and a water repellency rating above 75 using AATCC TM22 testing.

17. An absorbent article comprising:
an inner layer that rests against the skin of the user when the article is in use, said inner layer comprising a fabric of cellulosic fibers treated with at least one hydrophobic treatment selected from the group of:
blending with synthetic fibers;
leaving at least a portion of said cellulosic fibers unbleached so as to leave inherent lignin present; and
applying a hydrophobic agent to at least one surface of said cellulosic fibers, such agent being applied to at least one of a surface of said cellulosic fibers and a surface of said fabric, and applied at a solid loading percent of 15% or less.

18. The absorbent article of claim 17 wherein said at least one hydrophobic treatment includes applying, to at least one side of said cellulosic fibers, at least one compound selected from the group consisting of: silica-based nanoparticles; graphene-based coatings; TiO₂ (titanium dioxide) coatings; zinc oxide and zinc pyrithione coatings; copper oxide, copper gluconate, and copper sulfate coatings; epoxies; nanomaterial-based resins, polymers, and surfactants; lotus effect-inspired coatings; alkyl-based coatings; polymeric films; self-healing hydrophobic coatings; functionalized polymers with hydrophobic side chains; hydrophobic highly branched dendrimers; paraffins that are liquid at room temperature in water dispersible emulsions; and fatty acids.

19. An absorbent article comprising:
an outer layer that faces outwards from the skin of the user when the article is in use, said outer layer comprising a fabric of cellulosic fibers treated with at least one hydrophobic treatment selected from the group of:
blending with synthetic fibers;
leaving at least a portion of said cellulosic fibers unbleached so as to leave inherent lignin present; and
applying a hydrophobic agent to at least one surface of said cellulosic fibers, such agent being applied to at least one of a surface of said cellulosic fibers and a surface of said fabric, and applied at a solid loading percent of 2.75% or less.

20. The absorbent article in claim 19 wherein said at least one hydrophobic treatment includes applying, to at least one side of said cellulosic fibers, at least one compound selected from the group consisting of: silica-based nanoparticles; graphene-based coatings; TiO₂ (titanium dioxide) coatings; zinc oxide and zinc pyrithione coatings; copper oxide, copper gluconate, and copper sulfate coatings; epoxies; nanomaterial-based resins, polymers, and surfactants; lotus effect-inspired coatings; alkyl-based coatings; polymeric films; self-healing hydrophobic coatings; functionalized polymers with hydrophobic side chains; hydrophobic highly branched dendrimers; paraffins that are liquid at room temperature in water dispersible emulsions; and fatty acids.
